(19) United States
(12) Patent Application Publication
(10) Pub. No.: US 2017/0157323 A1
(43) Pub. Date: Jun. 8, 2017

(54) DEVICE-BASED SENSOR TRACKING FOR OPTIMIZING A CARDIOVASCULAR MEDICATION ADMINISTRATION PROTOCOL

(71) Applicant: Cardiac Pacemakers, Inc., St. Paul, MN (US)

(72) Inventors: Sunipa Saha, Shoreview, MN (US); Pramodsingh Hirasingh Thakur, Woodbury, MN (US)

(21) Appl. No.: 15/363,132

(22) Filed: Nov. 29, 2016

Related U.S. Application Data
(60) Provisional application No. 62/263,302, filed on Dec. 4, 2015.

(51) Int. Cl. A61M 5/172; A61N 1/365; G06F 19/00; A61N 1/39

(52) U.S. Cl. CPC A61M 5/1723; A61N 1/3956; A61N 1/36585; G06F 19/3456; G06F 19/3418; A61M 2230/04; A61M 2230/63; A61B 7/04

(57) ABSTRACT

In some examples, a method of adjusting a cardiovascular medication administration protocol for a subject includes receiving heart contractility surrogate information, obtained from a sensor configured to sense information about a heart of the subject, during a specified acute time period following an initiation or change in medication administration to the subject, determining, using a comparator circuit, whether the heart contractility surrogate information indicates an occurrence of at least a specified change in heart contractility during the specified acute time period following the initiation or change in medication administration to the subject, and adjusting the medication administration protocol using the determination of whether the heart contractility surrogate information indicates the occurrence of at least the specified change in heart contractility during the specified acute time period following the initiation or change in medication administration to the subject.

### CLAIM OF PRIORITY

This application claims the benefit of priority under 35 U.S.C. §119(e) of U.S. Provisional Patent Application Ser. No. 62/263,302, filed on Dec. 4, 2015, which is herein incorporated by reference in its entirety.

### TECHNICAL FIELD

This disclosure relates generally to systems, devices and methods for titrating cardiovascular medications.

### BACKGROUND

Implantable medical devices (IMDs) include devices designed to be implanted into a patient or subject. Some examples of these devices include cardiac function management (CFM) devices such as implantable pacemakers, implantable cardioverter defibrillators (ICDs), cardiac resynchronization therapy devices (CRTs), implantable diagnostic monitors, and devices that include a combination of such capabilities. The devices can be used to treat patients, e.g., heart failure patients, using electrical or other therapies, or to aid a physician or caregiver in patient diagnosis through internal monitoring of a patient's condition. The devices may include one or more electrodes in communication with one or more sense amplifiers to monitor electrical heart activity within a patient, and often include one or more sensors to monitor one or more other patient parameters. Other examples of implantable medical devices include implantable diagnostic devices, implantable drug delivery systems, or implantable devices with neural stimulation capability.

In heart failure (HF) patients, stroke volume can be compromised because of inefficient pumping of the heart. To compensate, the body can increase the heart rate to keep the cardiac output constant in spite of the decreasing stroke volume. Increased heart rates, however, can be undesirable. HF patients receiving implantable medical devices are often also receiving a pharmacological HF treatment therapy, including beta-blockers, angiotensin-converting-enzyme (ACE) inhibitors, and angiotensin II receptor blockers. Beta-blockers, such as carvedilol, can block β1 and β2 adrenergic receptors and can be used to decrease heart rate in HF patients. More particularly, beta-blocker therapy can be used to decrease heart rate by making adjustments that help HF patients compensate for any decrease in stroke volume by increasing heart contractility.

With a drug therapy, e.g., beta-blocker therapy, patients are typically started on an initial minimal dose, which can be increased, e.g., doubled, every two to four weeks, as tolerated. The dosage can be titrated up to a fixed target dose. Larger doses are often associated with better clinical outcomes.

### OVERVIEW

In general, this disclosure describes various techniques for improving and monitoring drug titrations based on information from device-based sensors tracking benefits and side-effects of the drug therapy.

In an aspect, this disclosure is directed towards a method of adjusting a cardiovascular medication administration protocol for a subject. The method includes receiving heart contractility surrogate information, obtained from a sensor configured to sense information about a heart of the subject, during a specified acute time period following an initiation or change in medication administration to the subject; determining, using a comparator circuit, whether the heart contractility surrogate information indicates an occurrence of at least a specified change in heart contractility during the specified acute time period following the initiation or change in medication administration to the subject; and adjusting the medication administration protocol using the determination of whether the heart contractility surrogate information indicates the occurrence of at least the specified change in heart contractility during the specified acute time period following the initiation or change in medication administration to the subject.

In another aspect, this disclosure is directed towards a method of adjusting a cardiovascular medication administration protocol for a subject. The method includes receiving S2 heart sound information, obtained from a sensor configured to sense information about a heart of the subject, during a specified acute time period following an initiation or change in medication administration to the subject; determining, using a comparator circuit, whether the S2 heart sound information indicates an occurrence of at least a specified change in S2 heart sound during the specified acute time period following the initiation or change in medication administration to the subject; and adjusting the medication administration protocol using the determination of whether the S2 heart sound information indicates the occurrence of at least the specified change in S2 heart sound during the specified acute time period following the initiation or change in medication administration to the subject.

In another aspect, this disclosure is directed towards a method of adjusting a cardiovascular medication administration protocol for a subject. The method includes receiving heart contractility surrogate information, obtained from a sensor configured to sense information about a heart of the subject, during a specified first acute time period following an initiation or change in medication administration to the subject; determining, using a comparator circuit, whether the heart contractility surrogate information indicates an occurrence of at least a specified change in heart contractility during the specified first acute time period following the initiation or change in medication administration to the subject; receiving S2 heart sound information, obtained from a sensor configured to sense information about a heart of the subject, during a specified second acute time period following the initiation or change in medication administration to the subject; determining, using the comparator circuit, whether the S2 heart sound information indicates an occurrence of at least a specified change in S2 heart sound during the specified second acute time period following the initiation or change in medication administration to the subject; and adjusting the medication administration protocol using: (1) the determination of whether the heart contractility surrogate information indicates the occurrence of at least the specified change in heart contractility during the specified first acute time period following the initiation or change in medication administration to the subject; and (2) the determination of whether the S2 heart sound information indicates the occurrence of at least the specified change in S2 heart sound during the specified second acute time period following the initiation or change in medication administration to the subject.

In another aspect, this disclosure is directed towards a method of adjusting a therapy protocol for a subject. The method includes receiving S1 heart sound amplitude under actual or simulated exercise information, obtained from a sensor configured to sense information about a heart of the subject; determining a contractile reserve indicator using a change in S1 heart sound amplitude; and adjusting the therapy protocol using the change in S1 heart sound.

This section is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is an illustration of portions of an example drug titration system that can use an implantable medical device and one or more implantable sensors to monitor and/or adjust a cardiovascular medication administration protocol for a subject, in accordance with various techniques of this disclosure.

FIG. 2 is a block diagram of an example of an IMD and implantable sensors that may be implemented in a drug titration system that can be used to implement various techniques of this disclosure.

FIG. 3 is a flow diagram of an example of a method for monitoring and/or adjusting a cardiovascular medication administration protocol for a subject, in accordance with various techniques of this disclosure.

FIG. 4 is a flow diagram of another example of a method for monitoring and/or adjusting a cardiovascular medication administration protocol for a subject, in accordance with various techniques of this disclosure.

FIG. 5 is an example of another method of adjusting a therapy protocol for a subject, in accordance with this disclosure.

FIG. 6 is an illustration of a system that includes an external device used to program parameters of an IMD.

### DETAILED DESCRIPTION

As mentioned above, cardiovascular medications, e.g., beta-blockers, angiotensin-converting-enzyme (ACE) inhibitors, and angiotensin II receptor blockers (ARBS) are important treatment options for patients with heart failure (HF). However, the administration of these drugs can be complicated because the proper dosages are difficult to determine for individual patients.

In general, this disclosure describes various techniques for monitoring and/or adjusting a cardiovascular medication administration protocol for a patient that can be executed by or in conjunction with the patient's implantable medical device (IMD), wearable device, or external device based on information from additional sensors that track the benefits and any side-effects of the protocol. These techniques can utilize capabilities in a medical device and one or more additional sensors to accomplish an appropriate medication administration protocol, e.g., drug titration.

More particularly, this disclosure describes various techniques for using sensor data, e.g., device-based sensors, to help determine what drug dosage is sufficient or optimal for a patient, as well as techniques for monitoring side effects using the sensors while staying optimally close to a target dose. In addition, this disclosure describes techniques for determining the likelihood that a patient will be a responder to the drug therapy based on a heart contractility reserve indicator.

The techniques described in this disclosure can advantageously be conducted with only minimal patient involvement. In addition, the techniques can allow remote monitoring of patients receiving HF medication, e.g., beta-blockers, ACE inhibitors, and ARBS, and can reduce the burden on patients, hospitals, and hospital staff for office visits for drug titrations. This can result in less inconvenience to the patient and less expense for the medical system. These techniques can increase accuracy compared to conventional techniques because sensors can track both the benefits and any patient side effects than when monitoring is only accomplished when the patient is seen in person by a medical professional.

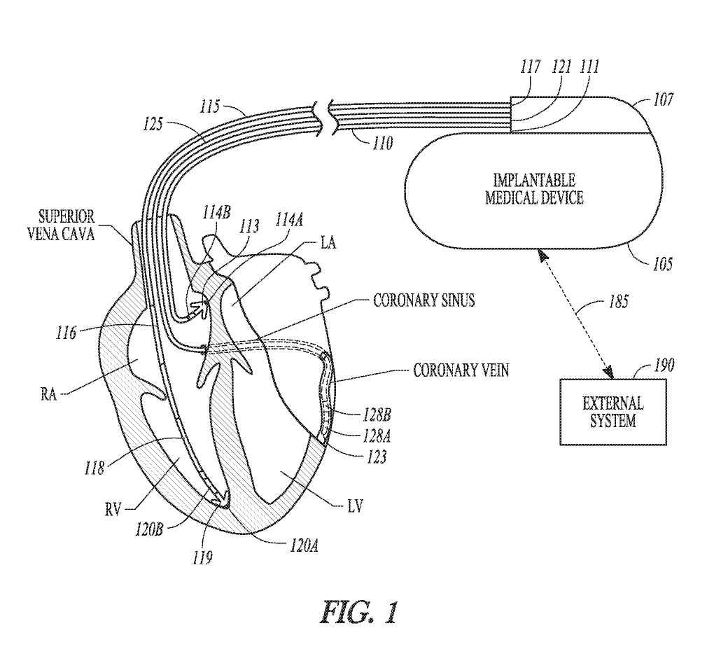

FIG. 1 is an illustration of portions of a drug titration system 100 that can use an IMD 105 and one or more implantable sensors to monitor and/or adjust a cardiovascular medication administration protocol for a subject, in accordance with various techniques of this disclosure. Examples of IMD 105 include, without limitation, a pacemaker, a cardioverter, a defibrillator, a cardiac resynchronization therapy (CRT) device, and other cardiac monitoring and therapy delivery devices, including cardiac devices that include or work in coordination with one or more neuro-stimulating devices, drugs, drug delivery systems, or other therapies. As one example, the system 100 shown is used to treat a cardiac arrhythmia. The IMD 105 typically includes an electronics unit coupled by one or more cardiac leads 110, 115, 125, to a heart of a patient or subject. The electronics unit of the IMD 105 typically includes components that are enclosed in a hermetically-sealed canister or “can.” The system 100 also typically includes an IMD programmer or other external system 190 that communicates one or more wireless signals 185 with the IMD 105, such as by using radio frequency (RF) or by one or more other telemetry methods.

The techniques of this disclosure are not limited to use with an implantable devices and/or implantable sensors. Rather, the techniques discussed herein could be used with implantable, wearable and/or external sensors, an implantable therapy device, an implantable diagnostic device, a wearable therapy device, a wearable diagnostic device, or other implantable or wearable or external devices. For purposes of conciseness, however, the techniques are generally described herein with reference to implantable sensors and devices, but the disclosure is not limited to such example implementations.

The example shown includes right atrial (RA) lead 110 having a proximal end 111 and a distal end 113. The proximal end 111 is coupled to a header connector 107 of the IMD 105. The distal end 113 is configured for placement in the RA in or near the atrial septum. The RA lead 110 may include a pair of bipolar electrodes, such as an RA tip electrode 114A and an RA ring electrode 114B. The RA electrodes 114A and 114B are incorporated into the lead body at distal end 113 for placement in or near the RA, and are each electrically coupled to IMD 105 through a conductor extending within the lead body. The RA lead is shown placed in the atrial septum, but the RA lead may be placed in or near the atrial appendage, the atrial free wall, or elsewhere.

The example shown also includes a right ventricular (RV) lead 115 having a proximal end 117 and a distal end 119. The proximal end 117 is coupled to a header connector 107. The distal end 119 is configured for placement in the RV. The RV lead 115 may include one or more of a proximal defibrillation electrode 116, a distal defibrillation electrode 118, an RV tip electrode 120A, and an RV ring electrode 120B. The defibrillation electrode 116 is generally incorporated into the lead body such as in a location suitable for supraventricular placement in the RA and/or the superior vena cava. The defibrillation electrode 118 is incorporated into the lead body near the distal end 119 such as for placement in the RV. The RV electrodes 120A and 120B may form a bipolar electrode pair and are generally incorporated into the lead body at distal end 119. The electrodes 116, 118, 120A, and 120B are each electrically coupled to IMD 105, such as through one or more conductors extending within the lead body. The proximal defibrillation electrode 116, distal defibrillation electrode 118, or an electrode formed on the can of IMD 105 allow for delivery of cardioversion or defibrillation pulses to the heart.

The RV tip electrode 120A, RV ring electrode 120B, or an electrode formed on the can of IMD 105 allow for sensing an RV electrogram signal representative of RV depolarizations and delivering RV pacing pulses. In some examples, the IMD includes a sense amplifier circuit to provide amplification and/or filtering of the sensed signal. RA tip electrode 114A, RA ring electrode 114B, or an electrode formed on the can of IMD 105 allow for sensing an RA electrogram signal representative of RA depolarizations and allow for delivering RA pacing pulses.

Sensing and pacing allows the IMD 105 to adjust timing of the heart chamber contractions. In some examples, the IMD 105 can adjust the timing of ventricular depolarizations with respect to the timing of atrial depolarizations by sensing electrical signals in the RA and pacing the RV at the desired atrial-ventricular (AV) delay time.

A left ventricular (LV) lead 125 can include a coronary pacing or sensing lead that includes an elongate lead body having a proximal end 121 and a distal end 123. The proximal end 121 is coupled to a header connector 107. A distal end 123 is configured for placement or insertion in the coronary vein. The LV lead 125 may include an LV tip electrode 128A and an LV ring electrode 128B. The distal portion of the LV lead 125 is configured for placement in the coronary sinus and coronary vein such that the LV electrodes 128A and 128B are placed in the coronary vein. The LV electrodes 128A and 128B may form a bipolar electrode pair and are typically incorporated into the lead body at distal end 123. Each can be electrically coupled to IMD 105 such as through one or more conductors extending within the lead body. LV tip electrode 128A, LV ring electrode 128B, or an electrode formed on the can of the IMD 105 allow for sensing an LV electrogram signal representative of LV depolarizations and delivering LV pacing pulses.

The IMDs may be configured with a variety of electrode arrangements, including transvenous, epicardial electrodes (i.e., intrathoracic electrodes), and/or subcutaneous, non-intrathoracic electrodes, including can, header, and indifferent electrodes, and subcutaneous array or lead electrodes (i.e., non-intrathoracic electrodes). Some IMDs are able to sense signals representative of cardiac depolarizations using electrodes without leads.

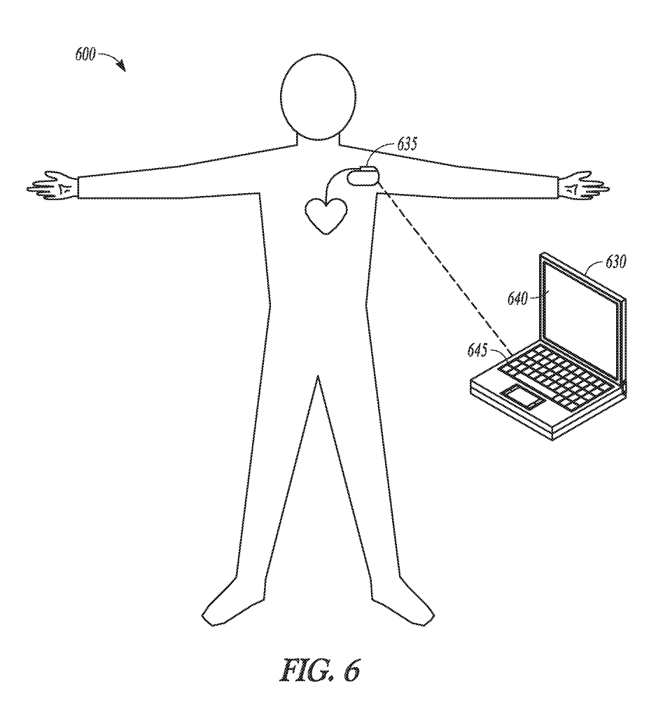

IMD 105 is further configured to communicate by telemetry with appropriately configured devices outside of the patient's body. For example, FIG. 6 illustrates a communication by telemetry from IMD 635 to an external patient interface device 630. Examples of patient interface devices include, but are not limited to, the LATITUDE® patient management system, the Model 2920 Programmer, and the Model 3120 Programmer, each available from Boston Scientific Corporation, Natick, Mass.

In some example configurations, IMD 105 may further include a drug reservoir (not depicted) that can controllably dispense a drug in response to device programming and sensor data.

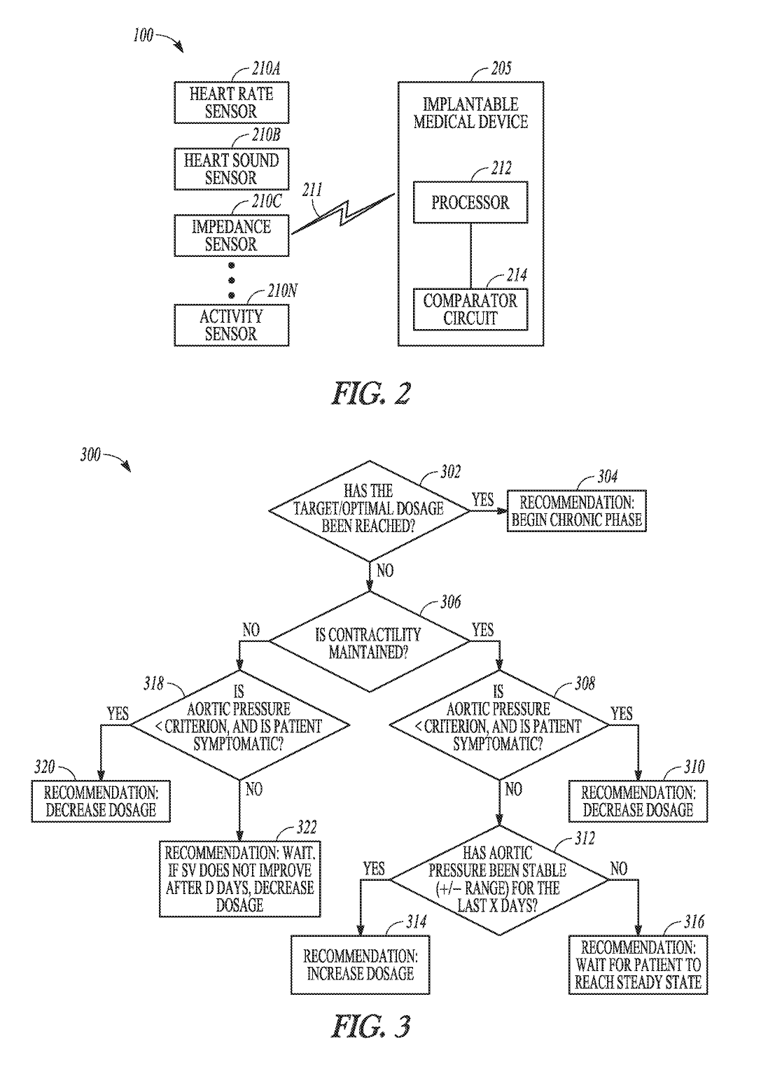

As indicated above, this disclosure describes various techniques for monitoring and/or adjusting a cardiovascular medication administration protocol for a patient that can be executed by or in conjunction with the patient's IMD, e.g., IMD 105 of FIG. 1, based on information from additional implantable sensors that can track the benefits and any side-effects of the protocol. Device programming of IMD 105 and sensor information received from the implantable sensors can optimize drug therapy in conjunction with CRT therapy, for example. An example of a system 100 having an implantable medical device and implantable sensors is depicted in FIG. 2.

It should be noted the techniques of this disclosure are not limited to implantable devices such as shown in FIG. 1. In other example implementations, various techniques of this disclosure can be performed with subcutaneous implantable cardioverter/defibrillator (S-ICD) devices as well as implantable diagnostic monitors.

FIG. 2 is a block diagram of an example of ID 205 and implantable sensors 210 that may be implemented in drug titration system 100 that can be used to implement various techniques of this disclosure. The system 100 of FIG. 1 includes IMD 205 and a plurality of sensors 210A-210N (collectively referred to in this disclosure as “sensors 210”) communicatively coupled to the IMD 205, e.g., via wireless links 211. IMD 205 represents an embodiment of IMD 105 and can include a processor 212 and a comparator circuit 214.

The processor 212 may include a microprocessor, a digital signal processor, application specific integrated circuit (ASIC), or other type of processor, interpreting or executing instructions in software or firmware. The comparator 214 can include, among other things, an electronic circuit comparator that can be constructed to perform the specific function of a comparison between a signal and a specified criterion, for example, or the comparator can be implemented as a portion of a general-purpose circuit that can be driven by a code instructing a portion of the general-purpose circuit to perform a comparison between the signal and the specified criterion.

Each of the sensors 210 provides a sensor signal that includes physiological information. The communicative coupling allows the processor 212 and the sensors 210 to communicate even though there may be intervening circuitry between the processor 212 and the sensors 210.

In some examples, the sensors 210 include an implantable heart sound sensor. Heart sounds are associated with mechanical vibrations from activity of a patient's heart and the flow of blood through the heart. Heart sounds recur with each cardiac cycle and are separated and classified according to the activity associated with the vibration. The first heart sound (S1) is the vibrational sound made by the heart during tensing of the mitral valve. The second heart sound (S2) marks the closing of the aortic valve and the beginning of diastole. The third heart sound (S3) and fourth heart sound (S4) are related to filling pressures of the left ventricle during diastole. A heart sound sensor produces an electrical signal which is representative of mechanical activity of a patient's heart. The heart sound sensor is disposed in a heart, near the heart, or in another location where the acoustic energy can be sensed. In some examples, the heart sound sensor includes an accelerometer disposed in or near a heart. In another example, the heart sound sensor includes an accelerometer disposed in the IMD. In another example, the heart sound sensor includes a microphone disposed in or near a heart.

Many types of physiological information can be included in a signal provided by a heart sound sensor. For example, the presence of an S3 heart sound may be an indication of elevated filling pressure. Thus, the development of, or a change in, an S3 heart sound may indicate a change in status of HF of the subject. An approach for monitoring heart sounds is found in Siejko et al., U.S. Pat. No. 7,972,275, titled “Method and Apparatus for Monitoring of Diastolic Hemodynamics,” filed Dec. 30, 2002, which is incorporated herein by reference in its entirety.

The sensors 210 can include a respiration sensor. An example of an implantable respiration sensor is an intra-thoracic impedance (Z) sensor. The signal provided by the Z-sensor provides physiological information that can be used to measure respiration parameters such as respiratory rate, tidal volume, minute respiration volume, and derived parameters such as the ratio of respiratory rate over tidal volume. The sensor signal provided by a Z-sensor can also provide information related to a change in fluid build-up in the thorax region of the subject. A decrease in impedance may indicate an increase in interstitial fluid build-up due to pulmonary edema. An approach to measuring thoracic impedance is described in Hartley et al., U.S. Pat. No. 6,076,015, “Rate Adaptive Cardiac Rhythm Management Device Using Transthoracic Impedance,” filed Feb. 27, 1998, which is incorporated herein by reference in its entirety.

In some examples, the sensors 210 can include an implantable patient activity sensor. An example of an implantable patient activity sensor is an accelerometer. The combination of a respiration sensor and an activity sensor, and/or the combination of a heart rate sensor and an activity sensor, is useful for monitoring a patient's physiological response to activity (PRA), such as to detect one or both of abnormal breathing and abnormal reflex sympathetic activation due to activity. The system 100 may include other types of sensors.

Using various techniques described in this disclosure, system 100 can monitor and/or adjust a cardiovascular medication administration protocol for a patient (also referred to as a subject) based on information from one or more of the sensors 210, which can track the benefits and any side-effects of the protocol. Various techniques for tracking the benefits of the administration of the cardiovascular medication, e.g., titration of beta-blockers, ACE inhibitors, and ARBS, will be described first and then various techniques for tracking the side effects of the administration of the cardiovascular medication will be described.

As mentioned above, in HF patients, stroke volume can be compromised, e.g., decrease, because of inefficient pumping of the heart. Heart failure patients can compensate the reduced SV by increasing their heart rate via an increased sympathetic drive. However, increased resting heart rate can be detrimental to these patients as it can stress the heart and can be associated with worse long term outcomes. Beta-blockers can work to slow down the patient's heart rate by blocking the beta receptors. Blocking of the beta-receptors also can result in an acute drop in contractility. In the immediate period following a beta-blocker therapy, this can actually lead to a decreased cardiac output, due to the taking away of the compensatory HR mechanisms to restore cardiac output in light of inefficient SV as well as reducing contractility, which can be important contributor of stroke volume.

Patients often do not feel well in the first few weeks (acute time frame) following the start of beta-blocker therapy. However, beyond the first few weeks some patients' hearts adapt by increasing the stroke volume and thus restoring the cardiac output. Patients that are able to augment their stroke volume and thus begin to tolerate beta-blocker mediated reduced HR are called responders. Patients that are not able to do so are called non-responders and have to either decrease these dosages to sub-optimal levels or have to be completely taken off the therapy.

Stroke volume is a function of heart contractility, afterload, and preload. Stroke volume can be increased by, for example, decreasing afterload, e.g., reducing the peripheral resistance, and/or increasing heart contractility. Contractility refers to the strength of a heart contraction. The primary mechanism of restoring stroke volume following a beta-blocker mediated decreased HR is via a gradual increase in contractility. Studies indicate that patients that have contractile reserve (e.g., an ability to increase contractility in response to sympathetic stimulation like exercise) can be able to augment their stroke volume via an increased contractility and thus respond to beta-blockers better than patients that do not have contractile reserve. Thus, contractile reserve can be a way to quantify or assess the total “benefit” that a patient can derive out of beta-blocker therapy. Patients with more contractile reserve can potentially derive more benefit out of a beta-blocker therapy. Note that this increase in contractility in responder patients occurs over a longer (chronic time frame). During an acute time frame, following the initiation of beta-blocker therapy, contractility often drops due to beta-blockade.

Contractile reserve can be assessed or quantified by measuring contractility increases in response to exercise or positive ionotropic challenges such as dobutamine stress test.

To track the benefits of the administration of the cardiovascular medication, the present inventors propose tracking a heart contractility indicator to determine whether contractility is changing; either decreasing over an acute time frame (e.g., 2-4 weeks) in response to initiating a beta-blocker therapy or increasing over chronic time frame, which can indicate an improvement in stroke volume.

Currently, patients are prescribed a fixed maximum target dosage, e.g., for metaprolol, carvedilol, etc., that was based on a clinical trial design. The goal in clinical practice is to get each patient as close to this target dose as possible in light of his/her response and side/effects.

The present inventors, however, have recognized that each patient likely has his or her own optimal target dosage based upon his/her physiology and contractile reserve. A one-shoe-fits-all solution of prescribing one common target dosage for all patients can be sub-optimal as it can over-prescribe the drug to a patient that may achieve the maximal benefit at lower dosage, and under-utilize the drug for a patient who may tolerate and respond to additional dosage beyond the maximum prescribed limit today.

The techniques of this disclosure can be used to assess the impact of a given dose of beta-blocker in terms of its acute impact on contractile reserve, which can be indicative of total potential benefit that a patient can theoretically derive out of beta-blocker therapy, and define an optimal beta-blocker dosage for a given individual based upon the dosage that best matches or masks the available contractile reserve in that patient. As described above, contractile reserve can be measured as the increase in contractility in response to an ionotropic/sympathetic challenge. If this challenge is followed by an increase in beta-blocker level, it can lead to an acute decrease in contractility. In other words, it can mask or match a portion of the patient's contractile reserve. The extent of this masking can increase with a further increase in beta-blocker level. At some point, any further increase in beta-blocker level will have a negligible effect on contractility, which is the β1-receptor saturation point. There are no additional β1-receptors with which to connect. At this point the current beta-blocker dosage can be closely matched with the patient's contractile reserve and any further increase in beta-blocker dosage will not produce any additional benefit to that particular patient. Therefore, the optimal patient dosage has been reached.

An example contractility indicator that can be used for tracking benefits is the maximum rate of change of left ventricle (LV) pressure (P), or maximum (max) LV dP/dt. Maximum LV dP/dt, however, cannot be directly measured using device sensors without an invasive pressure sensor within the LV which can be risky due to the potential of clots and stroke. As such, the present inventors propose using heart contractility surrogate information obtained from one or more sensors 210, e.g., implantable, wearable, and/or external sensors, configured to sense information about a heart of the subject to track the benefits of the drug therapy during a specified acute time period following an initiation or change in medication administration to the patient (or subject).

In an example implementation, heart contractility surrogate information can be provided by a pressure sensor in the coronary veins. It can be shown that pressure in the coronary veins on the outside of the LV chamber is proportional to LV pressure itself. Thus the maximum rate of change of the coronary venous pressure can be used as a surrogate for maximum LV dP/dt.

In another example implementation, heart contractility surrogate information can be provided by a combination of sensors 210. For example, heart contractility surrogate information can be provided by a combination of a heart rate (HR) sensor 210A and an activity sensor 210N, or PRA sensor. Increases in HR as a function of activity (exercise or simulated via dobutamine infusion or some other challenge drug) can be used as a surrogate of contractile reserve. Over an acute time period following an initiation or change in medication administration to the patient, e.g., titration of a beta-blocker, the magnitude of some elevated (peak or non-peak) exercise heart rate reduction or the slope of HR versus activity level (or the slope of HR versus dobutamine dosage level) reduction can be indicative of the masking of contractile reserve due to beta-blocker up-titration. At some point, any further increase in beta-blocker level will have a negligible effect on either the HR at a given elevated level of activity (exercise or simulated via dobutamine/inotrope infusion) or the slope of HR versus activity. Therefore, the optimal patient dosage has been reached.

In another example, heart contractility surrogate information can be provided by a combination of a heart sound sensor 210B and an activity sensor 210N. S1 heart sounds and contractility are highly correlated. Over an acute time period following an initiation or change in medication administration to the patient, e.g., titration of a beta-blocker, the magnitude of the S1 heart sound under actual or simulated exercise conditions can decrease as a beta-blocker level is increased. At some point, any further increase in beta-blocker level will have a negligible effect on S1 at a given activity (actual exercise) or dobutamine dosage level (simulated exercise). Therefore, the optimal patient dosage has been reached.

With respect heart contractility surrogate information, heart sounds may not be available during periods of high activity. However, heart rate can be readily tracked, even during periods of activity. On the other hand, heart rate may not be useful if the patient is atrial and ventricular paced all the time. Thus, use of a particular sensor's information can be based upon patient conditions. For example, heart rate and heart sounds can be used alone or in combination to obtain information during periods of rest and activity across various pacing conditions.

Referring again to FIG. 2, to adjust a cardiovascular medication administration protocol for a subject, IMD 205 and, in particular, the processor 212, can receive heart contractility surrogate information, obtained from one or more implantable sensors 210 configured to sense information about a heart of the subject, during a specified acute time period following an initiation or change in medication administration to the subject, e.g., a change in a beta-blocker level. In some examples, IMD 205 can receive heart rate under actual or simulated exercise information respectively obtained from the implantable heart rate sensor 210A and the implantable activity sensor 210N, e.g., accelerometer. Additionally or alternatively, IMD 205 can receive S1 heart sound amplitude under actual or simulated exercise information obtained from implantable heart sound sensor 210B and the implantable activity sensor 210N, e.g., accelerometer.

The comparator circuit 214 can compare the current heart contractility surrogate information to previously received heart contractility surrogate information and the processor 212 can determine whether the current heart contractility surrogate information indicates an occurrence of at least a specified change in heart contractility during the specified acute time period following the initiation or change in medication administration to the subject. For example. IMD 205 can determine whether heart contractility decreased by at least a specified amount during the specified acute time period following an increase in dosage levels (or uptitration) of the medication, e.g., a beta-blocker. In an example implementation, the comparator circuit 214 can compare a difference between the current heart contractility surrogate information to previously received heart contractility surrogate information to a specified criterion, e.g., threshold.

Using the determination of whether the heart contractility surrogate information indicates the occurrence of at least the specified change in heart contractility during the specified acute time period following the initiation or change in medication administration to the subject, the medication administration protocol can be adjusted. For example, if processor 212 determines that the heart contractility surrogate information indicates that heart contractility is essentially no longer changing, then the optimal dosage for that particular patient has been reached and the medication administration protocol can be adjusted to enter a chronic treatment phase. If, however, the processor 212 determines that the heart contractility surrogate information indicates that heart contractility is still changing, then the optimal dosage for that particular patient has not been reached and the medication administration protocol can be adjusted, e.g., increased, decreased, or the dosage can stay the same, as will be described in more detail below.

In addition to tracking the benefits of the cardiovascular medication administration protocol, this disclosure describes techniques for tracking any side-effects of the protocol, e.g., beta-blocker side-effects, while staying optimally close to the target dose. In some example implementations, side-effects, e.g., beta-blocker side-effects, can be tracked by receiving side-effect information reported by the patient. For example, the patient can report symptoms to a clinician, e.g., via the LATITUDE® patient management system.

In other example implementations, side-effect information can obtained, e.g., by IMD 205, from one or more implantable sensors 210. For example, side-effects, e.g., beta-blocker side-effects, can be tracked using blood pressure information of a patient. Instead of measuring blood pressure directly, however, S2 heart sound amplitude information, which is correlated with aortic pressure, can be obtained from an implantable heart sound sensor, e.g., heart sounds sensor 210B, and can used as a surrogate for blood pressure.

In an example implementation, the comparator circuit 214 can compare the aortic pressure surrogate information, e.g., S2 heart sound amplitude information obtained from implantable heart sound sensor 210B, to a specified criterion, e.g., a threshold, and if the processor 212 determines that the S2 amplitude information (aortic pressure surrogate) deviates from the specified criterion by at least a specified amount, the processor 212 can trigger an alert. In some examples, the processor 212 can monitor relative changes in S2 amplitude information to trigger an alert.

Additionally or alternatively, side-effects, e.g., beta-blocker side-effects, can be tracked using congestion and fluid retention information of a patient. Congestion and fluid retention information can be tracked using thoracic fluid status information received from at least one of the impedance sensor 210C and heart sound sensor 210B (S3 heart sound amplitude information). A decrease in impedance can indicate an increase in congestion and/or fluid retention. Although S3 heart sounds can occur normally in children and in young adults, S3 heart sounds can indicate congestion in adults.

The information from one or more sensors 210 tracking benefits and one or more sensors 210 tracking side-effects, e.g., beta-blocker side-effects, can be combined to drive therapy decisions and adjust a cardiovascular medication administration protocol for a subject, as described below with respect to FIG. 3. For example, a clinician can increase a dosage level, e.g., beta-blocker dosage level, decrease a dosage level, continue monitoring the patient before changing the dosage level, or if the target or optimal dosage has been reached, the clinician can begin a chronic phase of titration, e.g., beta-blocker titration.

FIG. 3 is a flow diagram of an example of a method 300 for monitoring and/or adjusting a cardiovascular medication administration protocol for a subject, in accordance with various techniques of this disclosure. More particularly, FIG. 3 depicts an example of a technique for monitoring a subject during an uptitration phase, e.g., where a clinician has initiated or changed a medication administration to the subject, of a beta-blocker therapy, for example. At block 302, the processor 212 can determine whether the subject has reached a target or optimal dosage of the medication, as described above. If the target or optimal dosage of the medication for the subject (“YES” branch of block 302), then the system 100 can recommend beginning a chronic phase of treatment at block 304.

If, however, the target or optimal dosage of the medication for the subject has not been reached (“NO” branch of block 302), the system 100 can determine if heart contractility (maximum LV dP/dt) of the subject has been maintained (block 306) using various techniques described above. For example, the system 100 can track heart contractility surrogate information obtained from one or more implantable sensors 210 configured to sense information about a heart of the subject to track the benefits of the drug therapy during a specified acute time period following an initiation or change in medication administration to the patient (or subject), e.g., using a combination of the HR sensor 210A and the activity sensor 210N (or PRA sensor), or using a combination of the heart sound sensor 210B and the activity sensor 210N. If the processor 212 determines that the heart contractility surrogate information indicates that heart contractility is essentially no longer changing and, as such, heart contractility is being maintained (“YES” branch of block 306), then the system 100 can track any side-effects at block 308.

At block 308, the system 100 can determine whether aortic pressure surrogate information is less than a specified criterion and if the patient is symptomatic. For example, the comparator circuit 214 can compare the aortic pressure surrogate information, e.g., S2 heart sound amplitude information obtained from implantable heart sound sensor 210B, to a specified criterion, e.g., a threshold. If the processor 212 determines that the S2 amplitude information (aortic pressure surrogate) is less than the specified criterion and that the patient is symptomatic, e.g., patient reported symptoms of feeling faint (“YES” branch of block 308), then the system 100 can recommend adjusting the medication administration protocol by decreasing the dosage, e.g., the beta-blocker dosage, at block 310. In response, the clinician, for example, can adjust the therapy.

If, however, the processor 212 determines that the S2 amplitude information (aortic pressure surrogate) is not less than the specified criterion, e.g., a threshold, and that the patient is not symptomatic (“NO” branch of block 308), then the processor 212 can determine if the S2 amplitude information (aortic pressure surrogate) has been generally stable, e.g., neither increased nor decreased by more than some specified percentage, for some specified time, e.g., one or more days (block 312). If the processor 212 determines that the S2 amplitude information (aortic pressure surrogate) has been stable (“YES” branch of block 312), then the system 100 can recommend adjusting the medication administration protocol by recommending increasing the dosage, e.g., the beta-blocker dosage, at block 314. In response, the clinician, for example, can adjust the therapy.

If, however, the processor 212 determines that the S2 amplitude information (aortic pressure surrogate) has not been stable (“NO” branch of block 312), then the system 100 can recommend adjusting the medication administration protocol by recommending waiting for the patient to reach a steady-state at block 316. In response, the clinician, for example, can adjust the therapy accordingly.

Returning to block 306, if the system 100 determines that the heart contractility has not improved (“NO” branch of block 306), e.g., if the processor 212 determines that the heart contractility surrogate information indicates that heart contractility is still changing, then at block 318, the system 100 can monitor any side effects to determine whether aortic pressure surrogate information is less than a specified criterion and if the patient is symptomatic. For example, the comparator circuit 214 can compare the aortic pressure surrogate information, e.g., S2 heart sound amplitude information obtained from implantable heart sound sensor 210B, to a specified criterion, e.g., a threshold. If the processor 212 determines that the S2 amplitude information (aortic pressure surrogate) is less than the specified criterion and that the patient is symptomatic, e.g., patient reported symptoms of feeling faint (“YES” branch of block 318), then the system 100 can recommend adjusting the medication administration protocol by decreasing the dosage, e.g., the beta-blocker dosage, at block 320. In response, the clinician, for example, can adjust the therapy.

If, however, the processor 212 determines that the S2 amplitude information (aortic pressure surrogate) is not less than the specified criterion, e.g., a threshold, and that the patient is not symptomatic (“NO” branch of block 318), then the system 100 can recommend waiting to adjust the medication administration protocol at block 322. If the contractility (as a surrogate for stroke volume) has not improved over time, e.g., one or more days, then the system 100 can recommend adjusting the medication administration protocol by recommending decreasing the dosage, e.g., the beta-blocker dosage, at block 322.

Referring to block 304, if the patient has begun the chronic phase of a cardiovascular medication administration protocol, the system 100 can track any side-effects of the protocol. For example, if the processor 212 determines that the S2 amplitude information (aortic pressure surrogate) is less than a specified criterion, e.g., a threshold, then the system 100 can set an alarm or other alert to notify the clinician, e.g., physician. In addition, in some example implementations, the processor 212 can modify one or more device parameters of IMD 205. For example, if the processor 212 determines that the aortic pressure is too low, the processor 212 can increase the pacing rate of pacing pulses delivered by the IMD 205, e.g., increase a lower rate limit (LRL), to prevent the patient from experiencing syncope, e.g., dizziness or fainting.

In addition to tracking any side effects using aortic pressure, if the patient has begun the chronic phase of a cardiovascular medication administration protocol (block 304), the system 100 can track any side-effects of the protocol by tracking congestion and fluid retention, e.g., using impedance and S3 heart sound measurements, as described above. For example, if the processor 212 determines that the impedance measurements from the impedance sensor 210C suggest fluid retention and/or congestion, e.g., the impedance measurements are below a specified criterion, then the system 100 can set an alarm or other alert to notify the clinician, e.g., physician. In addition, in some example implementations, the processor 212 can recommend adjusting a level of a diuretic protocol, e.g., uptitrate the level of diuretics.

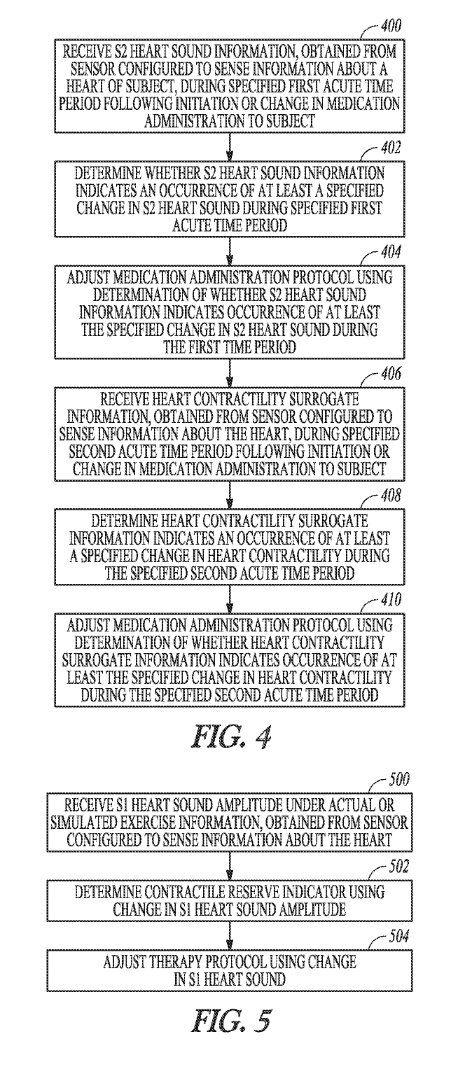

As described above, the present inventors have determined that heart contractility surrogate information during a specified acute time period following an initiation or change in medication administration to the subject can be used to adjust a cardiovascular medication administration protocol for a subject. The present inventors have also determined that S2 heart sound information, obtained from a sensor, e.g., implantable, wearable, or external, configured to sense information about a heart of the subject, during a specified acute time period following an initiation or change in medication administration to the subject can be used to adjust a cardiovascular medication administration protocol for a subject, as described below with respect to FIG. 4. In addition, in some example implementations, both heart contractility surrogate information and S2 heart sound information can be used to adjust a cardiovascular medication administration protocol for a subject.

Heart failure patients often receive angiotensin-converting-enzyme (ACE) inhibitors and/or angiotensin II receptor blockers (ARBS). ACE inhibitors and ARBS, however, can make a subject more hypotensive. For example, if ACE inhibitor dosage is increased, peripheral resistance can decrease, and the patient can be become hypotensive. S2 heart sound information can act as a blood pressure/hypotension measurement surrogate. Low S2 heart sound information can indicate low blood pressure. Thus, a patient's hypotension can be detected by the S2 heart sound information.

FIG. 4 is a flow diagram of another example of a method for monitoring and/or adjusting a cardiovascular medication administration protocol for a subject, in accordance with various techniques of this disclosure. More particularly, FIG. 4 depicts an example of a method of adjusting a cardiovascular medication administration protocol for a subject using S2 heart sound information. At block 400, to adjust a cardiovascular medication administration protocol for a subject, the processor 212 can receive S2 heart sound information, obtained from one or more sensors 210, e.g., implantable, wearable, or external, configured to sense information about a heart configured to sense information about a heart of the subject, e.g., heart sound sensor 210B, during a specified first acute time period following an initiation or change in medication administration to the subject. In some examples, the initiation or change in medication administration can include an initiation or change in administration of an ACE inhibitor and/or ARBS. In other examples, the initiation or change in medication administration can include an initiation or change in administration of a beta-blocker.

At block 402, the comparator circuit 214 can compare the current S2 heart sound information to previously received S2 heart sound information and the processor 212 can determine whether the current S2 heart sound information indicates an occurrence of at least a specified change in S2 heart sound, e.g., S2 heart sound increased or decreased with respect to a specified criterion, during the specified first acute time period following the initiation or change in medication administration to the subject. For example, IMD 205 can determine whether S2 heart sound decreased by at least a specified amount during the specified acute time period following an increase in dosage levels (or uptitration) of the medication, e.g., a beta-blocker, ACE inhibitor, or ARBS. In an example implementation, the comparator circuit 214 can compare a difference between the current S2 heart sound information to previously received S2 heart sound information to a specified criterion.

Using the determination of whether the S2 heart sound information indicates the occurrence of at least the specified change in S2 heart sound, e.g., S2 heart sound increased or decreased with respect to a specified criterion, during the specified first acute time period following the initiation or change in medication administration to the subject, the medication administration protocol can be adjusted (block 404). For example, if processor 212 determines that the S2 heart sound information meets a specified criterion, e.g., threshold, the system 100 can recommend adjusting the medication protocol by recommending one or more of the following: decreasing a dosing of at least one of an angiotensin-converting-enzyme (ACE) inhibitor and an angiotensin II receptor blocker in response to a decrease in S2 heart sound that meets a specified criterion; decreasing a beta-blocker dosing in response to a decrease in S2 heart sound that meets a specified criterion; and decreasing a diuretic dosing in response to a decreasing in S2 heart sound that meets a specified criterion. In response, the clinician, for example, can adjust the therapy. In some example implementations, if the processor 212 determines that the aortic pressure has fallen too low, the processor 212 can increase the pacing rate, e.g., increase a lower rate limit (LRL), to prevent the patient from experiencing syncope, e.g., dizziness or fainting, as mentioned above.

Heart failure patients may often receive multiple medications such as beta-blockers, diuretics, ACE inhibitors, ARBS, etc. at the same time under a cardiovascular medication protocol. Under such protocols, the techniques described with respect to FIG. 4 that use S2 heart sound information (as a blood pressure surrogate) can be combined with various techniques described above with respect to FIG. 3, for example, that use heart contractility surrogate information to track benefits using max LV dP/dt and track any side effects. Referring again to FIG. 4, the method shown optionally further includes blocks 406-410 that use heart contractility surrogate information to determine whether to adjust the medication administration protocol.

The steps described in blocks 406-410 were described in detail above and, for purposes of brevity and conciseness, will be described again briefly. During a specified second acute time period (where the specified second acute time period can be the same or different than the first specified time period in which S2 heart sound information is received), the system 100 can receive heart contractility surrogate information, obtained from one or more implantable sensors 210 configured to sense information about a heart of the subject, during a specified second acute time period following the initiation or change in medication administration to the subject (block 406). The comparator circuit 214 can compare the current heart contractility surrogate information to previously received heart contractility surrogate information and the processor 212 can determine whether the current heart contractility surrogate information indicates an occurrence of at least a specified change in heart contractility during the specified acute time period following the initiation or change in medication administration to the subject (block 408).

Using the determination of whether the heart contractility surrogate information indicates the occurrence of at least the specified change in heart contractility during the specified acute time period following the initiation or change in medication administration to the subject, the medication administration protocol can be adjusted (block 410).

In some example implementations of techniques for adjusting a cardiovascular medication administration protocol for a subject, the steps described in blocks 400-404 can be implemented without optional steps 406-410, as described above. In other example implementations of techniques for adjusting a cardiovascular medication administration protocol for a subject, the steps described in blocks 406-410 can be implemented without steps 400-404, as described above with respect to FIG. 2.

As mentioned above, the techniques described with respect to FIG. 4 that use S2 heart sound information (as a blood pressure surrogate) can be combined with various techniques described above that use heart contractility surrogate information to track any side effects. For example, in FIG. 4, if the cardiovascular medication that is being changed or initiated in block 400 includes a beta-blocker, the method can further include receiving beta-blocker side-effect information, during a specified second acute time period following the initiation or change in beta-blocker administration to the subject. Examples of receiving beta-blocker side-effect information were described in detail above and can include side-effect information reported by the subject and/or receiving the beta-blocker side-effect information obtained from a sensor. An example of receiving the beta-blocker side-effect information obtained from a sensor can include the processor 212 receiving S2 heart sound amplitude information obtained from implantable heart sound sensor 210B. Another example of receiving the beta-blocker side-effect information obtained from a sensor can include the processor 212 receiving thoracic fluid status information obtained from at least one of the impedance sensor 210C and the S3 heart sound amplitude sensor 210B.

The information from one or more sensors 210 tracking the beta-blocker side-effects can be combined to drive therapy decisions and adjust a cardiovascular medication administration protocol for a subject, as described above with respect to FIG. 3. For example, a clinician can increase a dosage level, e.g., beta-blocker dosage level, decrease a dosage level, continue monitoring the patient before changing the dosage level, or if the target or optimal dosage has been reached, begin a chronic phase of titration, e.g., beta-blocker titration. In examples in which the beta-blocker side-effect information includes receiving thoracic fluid status information obtained from at least one of an impedance sensor or an S3 heart sound amplitude sensor, adjusting the medication protocol can include at least one of the following: increasing a diuretic dosing in response to a decrease in S2 heart sound that meets a specified criterion, e.g., a threshold, and decreasing the beta-blocker dosing in response to a decrease in S2 heart sound that meets a specified criterion.

In addition to the various techniques described above for monitoring and/or adjusting a cardiovascular medication administration protocol for a patient, this disclosure describes techniques for determining the likelihood that a patient will be a responder to the drug therapy based on a contractile reserve indicator. The present inventors have determined that patients having a high contractility reserve indicator will likely respond to beta-blocker therapy while patients having a low contractility reserve indicator will likely not respond to beta-blocker therapy. By determining whether or not a patient will respond to therapy, these techniques provide a mechanism to screen patients before beta-blockers are titrated.

FIG. 5 is an example of another method of adjusting a therapy protocol for a subject, in accordance with this disclosure. The method of FIG. 5 includes the processor 212 receiving S1 heart sound amplitude under exercise information, obtained from at least one sensor 210, e.g., implantable, wearable, or external, configured to sense information about a heart of the subject (block 500). The exercise information can be the result of real (physical) exercise or simulated, e.g., using dobutamine as a stressor.

The processor 212 can determine a contractile reserve indicator using a change in the S1 heart sound amplitude (block 502). For example, if the processor 212 determines that the S1 heart sound amplitude under exercise information indicates that the S1 heart sound amplitude is increasing as the patient's activity level (real or simulated) is increasing, the processor 212 can determine that the patient has a high or sufficient contractile reserve and can generate a contractile reserve indicator. In some example implementations, the processor 212 can generate a contractile reserve indicator that indicates that the patient is either likely a responder or likely not a responder, e.g., binary. In other examples, the processor 212 can generate a contractile reserve indicator selected from one of several levels, e.g., more than two, based on a degree of response. If the processor 212 determines that the S1 heart sound amplitude under exercise information indicates that the S1 heart sound amplitude is not increasing as the patient's activity level (real or simulated) is increasing, the processor 212 can determine that the patient has a low or insufficient contractile reserve and can generate a contractile reserve indicator. Then, a therapy protocol can be adjusted, e.g., by a clinician or automatically by a device, using the change in in S1 heart sound. In some examples, the therapy protocol can include determining a responsiveness of the subject to the medication, e.g., beta-blocker, using the contractile reserve indicator. The determined responsiveness can, for example, be a score or other indicator that can be displayed, transmitted, or otherwise communicated to a clinician. The clinician can use the determined responsiveness of the subject to adjust the therapy protocol accordingly.

FIG. 6 is an illustration of a system 600 that includes an external device 630 used to program parameters of an IMD 635. The external device 630 includes a programming interface such as a display 640 and/or a keyboard 645 or computer mouse. The external device 630 communicates with the IMD 635 wirelessly. In some examples, the IMD 635 can communicate information described above to the external device 630 including, for example, sensor information and/or determined heart contractility surrogate information, and/or determined contractile reserve information.

The above detailed description includes references to the accompanying drawings, which form a part of the detailed description. The drawings show, by way of illustration, specific embodiments in which the invention can be practiced. These embodiments are also referred to herein as “examples.” All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

### Various Notes and Examples

Example 1 includes subject matter (such as a method, means for performing acts, machine readable medium (such as a computer-readable medium) including instructions that when performed by a machine cause the machine to performs acts, or an apparatus configured to perform) for adjusting a cardiovascular medication administration protocol for a subject, comprising: receiving heart contractility surrogate information, obtained from a sensor configured to sense information about a heart of the subject, during a specified acute time period following an initiation or change in medication administration to the subject; determining, using a comparator circuit, whether the heart contractility surrogate information indicates an occurrence of at least a specified change in heart contractility during the specified acute time period following the initiation or change in medication administration to the subject; and adjusting the medication administration protocol using the determination of whether the heart contractility surrogate information indicates the occurrence of at least the specified change in heart contractility during the specified acute time period following the initiation or change in medication administration to the subject.

In Example 2, the subject matter of Example 1 can optionally include, wherein the determining whether the heart contractility surrogate information indicates an occurrence of at least a specified change in heart contractility during the specified acute time period following the initiation or change in medication administration to the subject includes: determining whether heart contractility decreases by at least a specified amount during the specified acute time period following an uptitration of the medication, wherein the medication includes a beta-blocker.

In Example 3, the subject matter of one or more of Examples 1 and 2 can optionally include, wherein the receiving heart contractility surrogate information, obtained from a sensor configured to sense information about a heart of the subject, during a specified acute time period following the initiation or change in medication administration to the subject, comprises receiving heart rate under actual or simulated exercise information respectively obtained from an implantable heart rate sensor and an implantable accelerometer.

In Example 4, the subject matter of one or more of Examples 1-3 can optionally include, wherein the receiving heart contractility surrogate information, obtained from a sensor configured to sense information about a heart of the subject, during a specified acute time period following the initiation or change in medication administration to the subject, comprises receiving S1 heart sound amplitude under actual or simulated exercise information obtained from an implantable heart sound sensor and an implantable accelerometer.

In Example 5, the subject matter of one or more of Examples 1-4 can optionally include, wherein the medication includes a beta-blocker, and further comprising: receiving beta-blocker side-effect information, during a specified acute time period following the initiation or change in beta-blocker administration to the subject; and adjusting the beta-blocker medication administration protocol also using the beta-blocker side-effect information.

In Example 6, the subject matter of one or more of Examples 1-5 can optionally include, wherein receiving the beta-blocker side-effect information comprises receiving side-effect information reported by the subject.

In Example 7, the subject matter of one or more of Examples 1-6 can optionally include, wherein receiving the beta-blocker side-effect information comprises receiving the beta-blocker side-effect information obtained from a sensor.

In Example 8, the subject matter of one or more of Examples 1-7 can optionally include, wherein receiving beta-blocker side-effect information comprises receiving S2 heart sound amplitude information obtained from an implantable heart sound sensor.

In Example 9, the subject matter of one or more of Examples 1-8 can optionally include, wherein receiving beta-blocker side-effect information comprises receiving thoracic fluid status information obtained from at least one of an impedance sensor or an S3 heart sound amplitude sensor.

Example 10 includes subject matter (such as a method, means for performing acts, machine readable medium including instructions that when performed by a machine cause the machine to performs acts, or an apparatus configured to perform) for adjusting a cardiovascular medication administration protocol for a subject, comprising: receiving S2 heart sound information, obtained from a sensor configured to sense information about a heart of the subject, during a specified acute time period following an initiation or change in medication administration to the subject; determining, using a comparator circuit, whether the S2 heart sound information indicates an occurrence of at least a specified change in S2 heart sound during the specified acute time period following the initiation or change in medication administration to the subject; and adjusting the medication administration protocol using the determination of whether the S2 heart sound information indicates the occurrence of at least the specified change in S2 heart sound during the specified acute time period following the initiation or change in medication administration to the subject.

In Example 11, the subject matter of Example 10 can optionally include, wherein adjusting the medication protocol includes at least one of: decreasing a dosing of at least one of an angiotensin-converting-enzyme (ACE) inhibitor and an angiotensin II receptor blocker in response to a decrease in S2 heart sound that meets a specified criterion; decreasing a beta-blocker dosing in response to a decrease in S2 heart sound that meets a specified criterion; and decreasing a diuretic dosing in response to a decreasing in S2 heart sound that meets a specified criterion.

In Example 12, the subject matter of one or more of Examples 10 and 11 can optionally include, increasing a pacing rate in response to a decrease in S2 heart sound that meets a specified criterion.

In Example 13, the subject matter of one or more of Examples 10-12 can optionally include, wherein the specified acute time period is a specified first acute time period, the method further comprising: receiving heart contractility surrogate information, obtained from a sensor configured to sense information about a heart of the subject, during a specified second acute time period following the initiation or change in medication administration to the subject; determining, using a comparator circuit, whether the heart contractility surrogate information indicates an occurrence of at least a specified change in heart contractility during the specified second acute time period following the initiation or change in medication administration to the subject; and adjusting the medication administration protocol additionally using the determination of whether the heart contractility surrogate information indicates the occurrence of at least the specified change in heart contractility during the specified second acute time period following the initiation or change in medication administration to the subject.

In Example 14, the subject matter of one or more of Examples 10-13 can optionally include, wherein the receiving heart contractility surrogate information, obtained from a sensor configured to sense information about a heart of the subject, during the specified second acute time period following the initiation or change in medication administration to the subject, comprises receiving S1 heart sound amplitude under actual or simulated exercise information obtained from an implantable heart sound sensor and an implantable accelerometer.

In Example 15, the subject matter of one or more of Examples 10-14 can optionally include, wherein the specified acute time period is a specified first acute time period, and wherein the medication includes a beta-blocker, the method further comprising: receiving beta-blocker side-effect information, during a specified second acute time period following the initiation or change in beta-blocker administration to the subject; and adjusting the beta-blocker medication administration protocol also using the beta-blocker side-effect information.

In Example 16, the subject matter of one or more of Examples 10-15 can optionally include, wherein receiving the beta-blocker side-effect information comprises receiving side-effect information reported by the subject.

In Example 17, the subject matter of one or more of Examples 10-16 can optionally include, wherein receiving the beta-blocker side-effect information comprises receiving the beta-blocker side-effect information obtained from a sensor.

In Example 18, the subject matter of one or more of Examples 10-17 can optionally include, wherein receiving beta-blocker side-effect information comprises receiving S2 heart sound amplitude information obtained from an implantable heart sound sensor.

In Example 19, the subject matter of one or more of Examples 10-18 can optionally include, wherein receiving beta-blocker side-effect information comprises receiving thoracic fluid status information obtained from at least one of an impedance sensor or an S3 heart sound amplitude sensor, and wherein adjusting the medication protocol includes at least one of: increasing a diuretic dosing in response to a decreasing in S2 heart sound that meets a specified criterion; and decreasing a beta-blocker dosing in response to a decrease in S2 heart sound that meets a specified criterion.

Example 20 includes subject matter (such as a method, means for performing acts, machine readable medium including instructions that when performed by a machine cause the machine to performs acts, or an apparatus configured to perform) for adjusting a cardiovascular medication administration protocol for a subject, comprising: receiving heart contractility surrogate information, obtained from a sensor configured to sense information about a heart of the subject, during a specified first acute time period following an initiation or change in medication administration to the subject; determining, using a comparator circuit, whether the heart contractility surrogate information indicates an occurrence of at least a specified change in heart contractility during the specified first acute time period following the initiation or change in medication administration to the subject; receiving S2 heart sound information, obtained from a sensor configured to sense information about a heart of the subject, during a specified second acute time period following the initiation or change in medication administration to the subject; determining, using the comparator circuit, whether the S2 heart sound information indicates an occurrence of at least a specified change in S2 heart sound during the specified second acute time period following the initiation or change in medication administration to the subject; and adjusting the medication administration protocol using: (1) the determination of whether the heart contractility surrogate information indicates the occurrence of at least the specified change in heart contractility during the specified first acute time period following the initiation or change in medication administration to the subject; and (2) the determination of whether the S2 heart sound information indicates the occurrence of at least the specified change in S2 heart sound during the specified second acute time period following the initiation or change in medication administration to the subject.

Example 21 includes subject matter (such as a method, means for performing acts, machine readable medium including instructions that when performed by a machine cause the machine to performs acts, or an apparatus configured to perform) for adjusting a cardiovascular medication administration protocol for a subject, comprising: receiving S1 heart sound amplitude under actual or simulated exercise information, obtained from a sensor configured to sense information about a heart of the subject; determining a contractile reserve indicator using a change in S1 heart sound amplitude; and adjusting the therapy protocol using the change in S1 heart sound.

In Example 22, the subject of Example 21 can optionally include, wherein the therapy protocol includes determining a responsiveness of the subject to the medication.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods.

The code can form portions of computer program products. Further, the code can be tangibly stored on one or more volatile or non-volatile computer-readable media during execution or at other times. These computer-readable media can include, but are not limited to, hard disks, removable magnetic disks, removable optical disks (e.g., compact disks and digital video disks), magnetic cassettes, memory cards or sticks, random access memories (RAM's), read only memories (ROM's), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. A method of adjusting a cardiovascular medication administration protocol for a subject, the method comprising:
receiving heart contractility surrogate information, obtained from a sensor configured to sense information about a heart of the subject, during a specified acute time period following an initiation or change in medication administration to the subject;
determining, using a comparator circuit, whether the heart contractility surrogate information indicates an occurrence of at least a specified change in heart contractility during the specified acute time period following the initiation or change in medication administration to the subject; and
adjusting the medication administration protocol using the determination of whether the heart contractility surrogate information indicates the occurrence of at least the specified change in heart contractility during the specified acute time period following the initiation or change in medication administration to the subject.

2. The method of claim 1, wherein the determining whether the heart contractility surrogate information indicates an occurrence of at least a specified change in heart contractility during the specified acute time period following the initiation or change in medication administration to the subject includes:
determining whether heart contractility decreases by at least a specified amount during the specified acute time period following an uptitration of the medication, wherein the medication includes a beta-blocker.

3. The method of claim 1, wherein the receiving heart contractility surrogate information, obtained from a sensor configured to sense information about a heart of the subject, during a specified acute time period following the initiation or change in medication administration to the subject, comprises receiving heart rate under actual or simulated exercise information respectively obtained from an implantable heart rate sensor and an implantable accelerometer.

4. The method of claim 1, wherein the receiving heart contractility surrogate information, obtained from a sensor configured to sense information about a heart of the subject, during a specified acute time period following the initiation or change in medication administration to the subject, comprises receiving S1 heart sound amplitude under actual or simulated exercise information obtained from an implantable heart sound sensor and an implantable accelerometer.

5. The method of claim 1, wherein the medication includes a beta-blocker, and further comprising:
receiving beta-blocker side-effect information, during a specified acute time period following the initiation or change in beta-blocker administration to the subject; and
adjusting the beta-blocker medication administration protocol also using the beta-blocker side-effect information.

6. The method of claim 5, wherein receiving the beta-blocker side-effect information comprises receiving side-effect information reported by the subject.

7. The method of claim 5, wherein receiving the beta-blocker side-effect information comprises receiving the beta-blocker side-effect information obtained from a sensor.

8. The method of claim 7, wherein receiving beta-blocker side-effect information comprises receiving S2 heart sound amplitude information obtained from an implantable heart sound sensor.

9. The method of claim 8, wherein receiving beta-blocker side-effect information comprises receiving thoracic fluid status information obtained from at least one of an impedance sensor or an S3 heart sound amplitude sensor.

10. A method of adjusting a cardiovascular medication administration protocol for a subject, the method comprising:
receiving S2 heart sound information, obtained from a sensor configured to sense information about a heart of the subject, during a specified acute time period following an initiation or change in medication administration to the subject;
determining, using a comparator circuit, whether the S2 heart sound information indicates an occurrence of at least a specified change in S2 heart sound during the specified acute time period following the initiation or change in medication administration to the subject; and
adjusting the medication administration protocol using the determination of whether the S2 heart sound information indicates the occurrence of at least the specified change in S2 heart sound during the specified acute time period following the initiation or change in medication administration to the subject.

11. The method of claim 10, wherein adjusting the medication protocol includes at least one of:
decreasing a dosing of at least one of an angiotensin-converting-enzyme (ACE) inhibitor and an angiotensin II receptor blocker in response to a decrease in S2 heart sound that meets a specified criterion;
decreasing a beta-blocker dosing in response to a decrease in S2 heart sound that meets a specified criterion; and
decreasing a diuretic dosing in response to a decreasing in S2 heart sound that meets a specified criterion.

12. The method of claim 11, further comprising increasing a pacing rate in response to a decrease in S2 heart sound that meets a specified criterion.

13. The method of claim 10, wherein the specified acute time period is a specified first acute time period, the method further comprising:
receiving heart contractility surrogate information, obtained from a sensor configured to sense information about a heart of the subject, during a specified second acute time period following the initiation or change in medication administration to the subject;
determining, using a comparator circuit, whether the heart contractility surrogate information indicates an occurrence of at least a specified change in heart contractility during the specified second acute time period following the initiation or change in medication administration to the subject; and
adjusting the medication administration protocol additionally using the determination of whether the heart contractility surrogate information indicates the occurrence of at least the specified change in heart contractility during the specified second acute time period following the initiation or change in medication administration to the subject

14. The method of claim 13, wherein the receiving heart contractility surrogate information, obtained from a sensor configured to sense information about a heart of the subject, during the specified second acute time period following the initiation or change in medication administration to the subject, comprises receiving S1 heart sound amplitude under actual or simulated exercise information obtained from an implantable heart sound sensor and an implantable accelerometer.

15. The method of claim 10, wherein the specified acute time period is a specified first acute time period, and wherein the medication includes a beta-blocker, the method further comprising:
receiving beta-blocker side-effect information, during a specified second acute time period following the initiation or change in beta-blocker administration to the subject; and
adjusting the beta-blocker medication administration protocol also using the beta-blocker side-effect information.

16. The method of claim 15, wherein receiving the beta-blocker side-effect information comprises receiving side-effect information reported by the subject.

17. The method of claim 15, wherein receiving the beta-blocker side-effect information comprises receiving the beta-blocker side-effect information obtained from a sensor.

18. The method of claim 17, wherein receiving beta-blocker side-effect information comprises receiving S2 heart sound amplitude information obtained from an implantable heart sound sensor.

19. The method of claim 17, wherein receiving beta-blocker side-effect information comprises receiving thoracic fluid status information obtained from at least one of an impedance sensor or an S3 heart sound amplitude sensor, and
wherein adjusting the medication protocol includes at least one of:
increasing a diuretic dosing in response to a decreasing in S2 heart sound that meets a specified criterion; and
decreasing a beta-blocker dosing in response to a decrease in S2 heart sound that meets a specified criterion.

20. A method of adjusting a cardiovascular medication administration protocol for a subject, the method comprising:
receiving heart contractility surrogate information, obtained from a sensor configured to sense information about a heart of the subject, during a specified first acute time period following an initiation or change in medication administration to the subject;
determining, using a comparator circuit, whether the heart contractility surrogate information indicates an occurrence of at least a specified change in heart contractility during the specified first acute time period following the initiation or change in medication administration to the subject;
receiving S2 heart sound information, obtained from a sensor configured to sense information about a heart of the subject, during a specified second acute time period following the initiation or change in medication administration to the subject;
determining, using the comparator circuit, whether the S2 heart sound information indicates an occurrence of at least a specified change in S2 heart sound during the specified second acute time period following the initiation or change in medication administration to the subject; and
adjusting the medication administration protocol using: (1) the determination of whether the heart contractility surrogate information indicates the occurrence of at least the specified change in heart contractility during the specified first acute time period following the initiation or change in medication administration to the subject; and (2) the determination of whether the S2 heart sound information indicates the occurrence of at least the specified change in S2 heart sound during the specified second acute time period following the initiation or change in medication administration to the subject.
